(19) United States
(12) Patent Application Publication
(10) Pub. No.: US 2026/0096818 A1
(43) Pub. Date: Apr. 9, 2026

(54) Inflatable Colorectal Outer Wall Constriction Device

(71) Applicants: Jing Lu, Rancho Cucamonga, CA (US); Yong Zhang, Xi'an (CN); Jie Shao, Xi'an (CN); Zicai Zhu, Xi'an (CN)

(72) Inventors: Jing Lu, Rancho Cucamonga, CA (US); Yong Zhang, Xi'an (CN); Jie Shao, Xi'an (CN); Zicai Zhu, Xi'an (CN)

(21) Appl. No.: 18/906,176

(22) Filed: Oct. 4, 2024

(51) Int. Cl. A61B 17/11; A61B 17/22

(52) U.S. Cl. CPC A61B 17/1114; A61B 2017/111; A61B 2017/22054

(57) ABSTRACT

This application relates to an inflatable colorectal outer wall constriction device designed for use in post-surgical anastomotic protection. The device consists of a support rod, a coiled inflatable tube, a head inflatable tube, a coiled inflatable balloon, and a head inflatable balloon. One end of the support rod includes an inflation port, while the other end is connected to both the coiled and head inflatable balloons. The tubes run through the hollow support rod, and when inflated, the coiled balloon curls into a ring while the head balloon expands, fitting securely into a slot near the coiled balloon. This configuration allows the device to apply pressure and form a tight seal between the outer intestinal wall and a fecal diversion device, preventing leakage. After the anastomosis has healed, the device can be deflated and easily removed, ensuring the proper protection of the surgical site during the healing process.

### BACKGROUND OF THE INVENTION

The embodiments of this application relate to the field of colorectal surgical instruments, specifically to an inflatable colorectal outer wall constriction device.

Postoperative colorectal surgeries are prone to anastomotic site contamination by intestinal contents, leading to infections, slow healing, and severe complications such as anastomotic leakage, especially after low rectal anastomosis surgeries. The primary method to prevent anastomotic site infection is preventive diversion, timely removing residual intestinal contents and avoiding contact with the anastomotic site.

Current technical solutions commonly use simple support anal tubes or intestinal decompression tubes for drainage. For instance, patent CN216061445U discloses an intestinal decompression tube used for anal decompression and anastomotic leakage prevention after mid-to-low rectal surgery, utilizing a central hole balloon and drainage tube to expel intestinal contents. However, this device lacks an outer intestinal constriction mechanism at the front end, making the decompression tube prone to slipping. Moreover, the internal decompression tube's sealing against the intestinal wall is poor, leading to residual feces leaking and contaminating the anastomotic site.

Thus, there is an urgent clinical need for an outer intestinal wall constriction device to apply a certain constraint on the intestine, ensure the sealing of the internal fecal diversion device, facilitate its removal, and provide reliable and safe constriction and expansion actions.

### SUMMARY OF THE INVENTION

The embodiments of this application provide an inflatable colorectal outer wall constriction device, which can be used for the tight sealing of the outer intestinal wall in postoperative colorectal anastomotic protection devices, and can be deflated and removed after the anastomosis has healed.

To address the aforementioned technical issues, the embodiments of this application provide an inflatable colorectal outer wall constriction device, comprising: a support rod, a coiled inflatable tube, a head inflatable tube, a coiled inflatable balloon, and a head inflatable balloon. One end of the support rod is provided with an inflation port, and the other end is sequentially connected to the coiled inflatable balloon and the head inflatable balloon. In use, the inflation port is located outside the body. The inflation port includes a first inflation port and a second inflation port. The support rod has a hollow conduit, with the coiled inflatable tube and the head inflatable tube located within it. The first inflation port is connected to the coiled inflatable balloon via the coiled inflatable tube, and the second inflation port is connected to the head inflatable balloon via the head inflatable tube. The end of the support rod near the coiled inflatable balloon is provided with a slot that matches the head inflatable balloon. When the first inflation port is inflated, the coiled inflatable balloon curls into a ring. When the second inflation port is inflated, the head inflatable balloon expands, securing the expanded head inflatable balloon into the slot, thereby applying a certain constraint to the outer intestinal wall and ensuring that the fecal diversion device is tightly sealed against the inner wall of the intestinal cavity.

In some exemplary embodiments, the support rod is connected to the coiled inflatable balloon at a balloon connection part, which is used to connect the support rod and the coiled inflatable balloon.

In some exemplary embodiments, the side wall of the coiled inflatable balloon is equipped with a first elastic metal rod and a second elastic metal rod. When inflated, the coiled inflatable balloon curls into a ring, and when deflated, it naturally unfolds under the action of the first and second elastic metal rods.

In some exemplary embodiments, the inner wall of the coiled inflatable balloon includes a first inner wall and a second inner wall. The thickness of the first inner wall is greater than that of the second inner wall, and the first and second elastic metal rods are embedded in the first inner wall.

In some exemplary embodiments, the head inflatable tube passes through the first inner wall and connects to the head inflatable balloon, and the head inflatable tube transversely passes between the first and second elastic metal rods, which are symmetrically arranged with respect to the head inflatable tube.

In some exemplary embodiments, the cross-section of the first and second elastic metal rods is elliptical, and they can only bend and recover in one direction, ensuring the precise curling of the coiled inflatable balloon when inflated and its return to a straight line when deflated.

In some exemplary embodiments, the outer diameter of the support rod is greater than the outer diameter of the coiled inflatable balloon, and the outer diameter of the coiled inflatable balloon is greater than the outer diameter of the head inflatable balloon.

In some exemplary embodiments, the fecal diversion device comprises an inflation washing unit, a first balloon air tube, a cleaning tube, a second balloon air tube, an intestinal protective membrane, a first support balloon, and a second support balloon. The intestinal protective membrane has a hollow conduit, with one end of the first balloon air tube, cleaning tube, and second balloon air tube located within the hollow conduit, and the other end located outside the intestinal protective membrane and the intestinal cavity, with the other end connected to the opening of the inflation washing unit.

In some exemplary embodiments, the first and second support balloons are spaced apart at the end of the intestinal protective membrane away from the inflation washing unit. The first support balloon is connected to the first balloon air tube, and the second support balloon is connected to the second balloon air tube.

In some exemplary embodiments, the inflatable colorectal outer wall constriction device is located between the first and second support balloons. The first and second support balloons are constrained by the inflatable colorectal outer wall constriction device to ensure tight sealing with the intestinal inner wall.

The technical solutions provided by the embodiments of this application have at least the following advantages:

The embodiments of this application provide an inflatable colorectal outer wall constriction device, comprising: a support rod, a coiled inflatable tube, a head inflatable tube, a coiled inflatable balloon, and a head inflatable balloon. One end of the support rod is provided with an inflation port, and the other end is sequentially connected to the coiled inflatable balloon and the head inflatable balloon. In use, the inflation port is located outside the body. The inflation port includes a first inflation port and a second inflation port. The support rod has a hollow conduit, with the coiled inflatable tube and the head inflatable tube located within it. The first inflation port is connected to the coiled inflatable balloon via the coiled inflatable tube, and the second inflation port is connected to the head inflatable balloon via the head inflatable tube. The end of the support rod near the coiled inflatable balloon is provided with a slot that matches the head inflatable balloon. When the first inflation port is inflated, the coiled inflatable balloon curls into a ring. When the second inflation port is inflated, the head inflatable balloon expands, securing the expanded head inflatable balloon into the slot, thereby applying a certain constraint to the outer intestinal wall and ensuring that the fecal diversion device is tightly sealed against the inner wall of the intestinal cavity.

The inflatable colorectal outer wall constriction device provided by this application achieves curling and locking actions through a two-stage balloon system, with wrapping force adjustable via inflation pressure. It unfolds during removal due to internal elastic metal rods, providing better outer wall pressure and sealing compared to existing solutions, reducing the likelihood of residual feces leaking and contaminating the anastomotic site. The curling and unfolding actions can be performed externally, ensuring safety and reliability. The inflatable constriction device features a simple structure, no special material requirements, and lower cost, offering better economic benefits compared to complex mechanical constriction mechanisms or absorbable material solutions.

### BRIEF DESCRIPTION OF DRAWINGS

T One or more embodiments are illustratively described through the corresponding figures in the drawings. These illustrative descriptions do not constitute limitations on the embodiments unless specifically stated otherwise. The figures in the drawings do not constitute scale limitations.

FIG. 1 is a schematic diagram showing the main structural components of an inflatable colorectal outer wall constriction device according to one embodiment of the present application.

FIG. 2 is a schematic diagram showing the structure of the inflatable colorectal outer wall constriction device in use according to one embodiment of the present application.

FIG. 3 is a side view of the inflatable colorectal outer wall constriction device according to one embodiment of the present application.

FIG. 4 is a schematic cross-sectional view of the coiled inflatable balloon according to one embodiment of the present application.

FIG. 5 is a schematic cross-sectional view of the head inflatable balloon according to one embodiment of the present application.

FIG. 6 is a schematic diagram showing the annular structure during inflation and constriction according to one embodiment of the present application.

FIG. 7 is a schematic diagram showing the structure of the fecal diversion device according to one embodiment of the present application.

Reference Numerals: 1—Intestinal cavity, 2—Fecal diversion device, 3—Inflatable colorectal outer wall constriction device, 11—Anastomotic site, 301—Inflation port, 302—Support rod, 303—Coiled inflatable tube, 304—Head inflatable tube, 305—Slot, 306—Balloon connection part, 307—Coiled inflatable balloon, 308—Head inflatable balloon, 309—First elastic metal rod, 310—Second elastic metal rod.

### DETAILED DESCRIPTION OF THE INVENTION

From the background, it is known that the existing intestinal anastomosis protection and fecal diversion devices lack external intestinal constriction mechanisms at the front end, leading to easy slippage of the decompression tube and poor sealing between the decompression tube and the intestinal wall, resulting in residual feces spilling and contaminating the anastomosis site.

To address the above technical issues, embodiments of the present application provide an inflatable colorectal outer wall constriction device, which includes: a support rod, a coiled inflatable tube, a head inflatable tube, a coiled inflatable balloon, and a head inflatable balloon. One end of the support rod is provided with an inflation port, while the other end is sequentially connected to the coiled inflatable balloon and the head inflatable balloon. During use, the inflation port is located outside the body. The inflation port includes a first inflation port and a second inflation port. The support rod has a hollow conduit, within which the coiled inflatable tube and the head inflatable tube are located. The first inflation port is connected to the coiled inflatable balloon via the coiled inflatable tube, and the second inflation port is connected to the head inflatable balloon via the head inflatable tube. The end of the support rod near the coiled inflatable balloon is provided with a slot that matches the head inflatable balloon. When air is pumped into the first inflation port, the coiled inflatable balloon can coil into an annular shape. When air is pumped into the second inflation port, the head inflatable balloon expands and locks into the slot, thereby applying a certain constraint to the outer wall of the intestine to ensure a tight seal between the fecal diversion device and the inner wall of the intestinal cavity.

This inflatable colorectal outer wall constriction device, provided by the present application, can be used for the external intestinal wall constriction and sealing of an anastomosis protection device post colorectal surgery. The device can be deflated and removed after the anastomosis has healed.

Below, the embodiments of the present application will be described in detail with reference to the accompanying drawings. However, it should be understood by those skilled in the art that numerous technical details have been proposed in the embodiments to enable better understanding of the present application. Even without these technical details and various changes and modifications based on the following embodiments, the technical solutions claimed in the present application can still be realized.

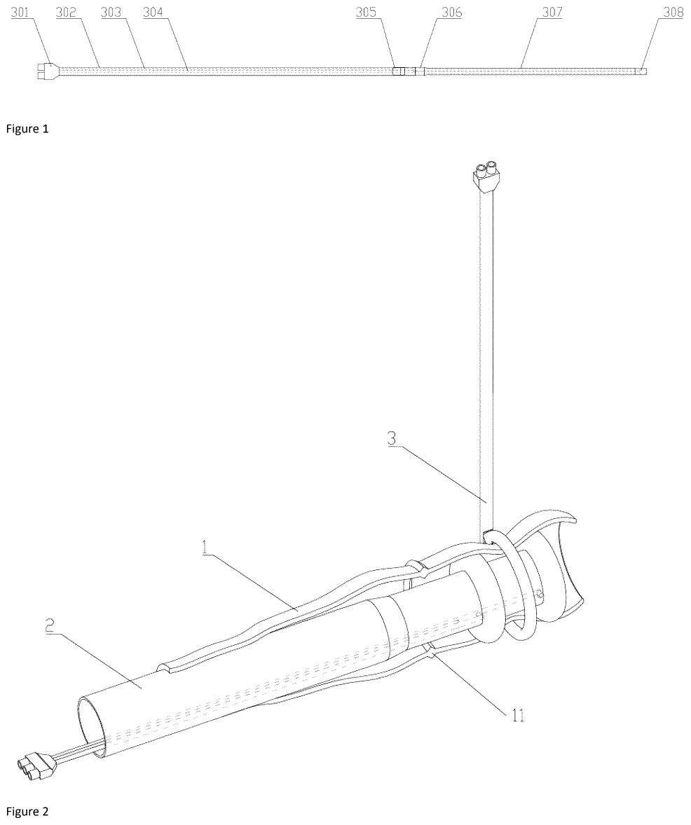

Referring to FIG. 1, an embodiment of the present application provides an inflatable colorectal outer wall constriction device, which includes: a support rod (302), a coiled inflatable tube (303), a head inflatable tube (304), a coiled inflatable balloon (307), and a head inflatable balloon (308). One end of the support rod (302) is provided with an inflation port (301), while the other end is sequentially connected to the coiled inflatable balloon (307) and the head inflatable balloon (308). During use, the inflation port (301) is located outside the body. The inflation port (301) includes a first inflation port and a second inflation port. The support rod (302) has a hollow conduit, within which the coiled inflatable tube (303) and the head inflatable tube (304) are located. The first inflation port is connected to the coiled inflatable balloon (307) via the coiled inflatable tube (303), and the second inflation port is connected to the head inflatable balloon (308) via the head inflatable tube (304). The end of the support rod (302) near the coiled inflatable balloon (307) is provided with a slot (305) that matches the head inflatable balloon (308). When air is pumped into the first inflation port, the coiled inflatable balloon (307) can coil into an annular shape. When air is pumped into the second inflation port, the head inflatable balloon (308) expands and locks into the slot (305), thereby applying a certain constraint to the outer wall of the intestine to ensure a tight seal between the fecal diversion device (2) and the inner wall of the intestinal cavity (1).

As shown in FIG. 2, the main structural schematic of the inflatable colorectal outer wall constriction device (3) provided in the present application, used in conjunction with the fecal diversion device (2), shows the inflatable colorectal outer wall constriction device (3) in a constricted state positioned between the two balloons of the fecal diversion device (2)'s front portion, applying a certain constraint to the outer wall of the intestine to ensure a tight seal between the fecal diversion device (2) and the inner wall of the intestinal cavity (1).

The inflatable colorectal outer wall constriction device provided by the present application can be used for external intestinal wall constriction in an anastomosis protection device post colorectal surgery. When used, the coiled inflatable balloon (307) is inflated to constrict the fecal diversion device (2) against the outer wall of the intestine. During removal, the device is deflated and taken out. The constriction force can be adjusted by the inflation pressure, making it easier to operate and adjust compared to existing solutions. It is structurally simple, easy to dismantle, safe, and reliable. The coiling and stretching actions can be completed through external manipulation, and it is also economically efficient.

Referring again to FIG. 1, the inflation port (301) is located at the very front end and positioned outside the body during use. The first inflation port of the inflation port (301) is connected to the coiled inflatable balloon (307) via the coiled inflatable tube (303), and the second inflation port of the inflation port (301) is connected to the head inflatable balloon (308) via the head inflatable tube (304). The slot (305) is located at the end of the support rod (302) near the inflatable balloons and is used to lock the head inflatable balloon (308) during constriction, ensuring reliable annular constriction.

Referring again to FIG. 1, in some embodiments, the connection between the support rod (302) and the coiled inflatable balloon (307) is provided with a balloon connection part (306), which is used to connect the support rod (302) with the coiled inflatable balloon (307).

Referring to FIG. 4, in some embodiments, the side wall of the coiled inflatable balloon (307) is provided with a first elastic metal rod (309) and a second elastic metal rod (310). When inflated, the coiled inflatable balloon (307) coils into an annular shape, and when deflated, the coiled inflatable balloon (307) naturally uncoils under the action of the first elastic metal rod (309) and the second elastic metal rod (310).

It should be noted that the balloon connection part (306) is located at the end of the support rod (302) and is used to connect the coiled section. As shown in FIG. 1, the coiled section consists of the coiled inflatable balloon (307), the head inflatable balloon (308), the first elastic metal rod (309), and the second elastic metal rod (310). The head inflatable balloon (308) is positioned at the very front end and is used for locking fixation. The first elastic metal rod (309) and the second elastic metal rod (310) are embedded in the side wall of the coiled inflatable balloon (307). When inflated, they coil into an annular shape, and when deflated, they naturally uncoil.

Referring again to FIG. 4, in some embodiments, the inner wall of the coiled inflatable balloon (307) includes a first inner wall and a second inner wall, with the first inner wall being thicker than the second inner wall. The first elastic metal rod (309) and the second elastic metal rod (310) are embedded in the first inner wall. Specifically, the design of the side walls of the coiled inflatable balloon (307) has different thicknesses, with the inner wall being divided into the first inner wall and the second inner wall. The first inner wall (the thicker side) has the first elastic metal rod (309), the second elastic metal rod (310), and the head inflatable tube (304) embedded within it. The first elastic metal rod (309) and the second elastic metal rod (310) are symmetrically arranged, with the head inflatable tube (304) passing horizontally between them.

Referring again to FIG. 4, in some embodiments, the head inflatable tube (304) penetrates through the first inner wall and connects to the head inflatable balloon (308), with the head inflatable tube (304) passing horizontally between the first elastic metal rod (309) and the second elastic metal rod (310). The first elastic metal rod (309) and the second elastic metal rod (310) are symmetrically arranged around the head inflatable tube (304).

In some embodiments, the cross-section of the first elastic metal rod (309) and the second elastic metal rod (310) is oval, allowing the first elastic metal rod (309) and the second elastic metal rod (310) to bend and recover in only one direction. This ensures that the coiled inflatable balloon (307) precisely coils into an annular shape when inflated and returns to a straight line when deflated.

Referring to FIG. 5, in some embodiments, the thickness of both sides of the head inflatable balloon (308) is uniform. The balloon expands when inflated, locking into the slot (305) of the support rod (302).

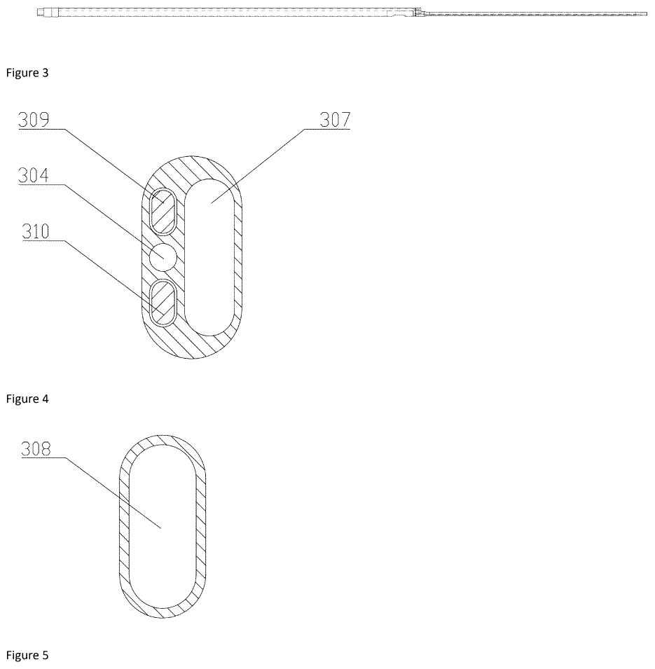

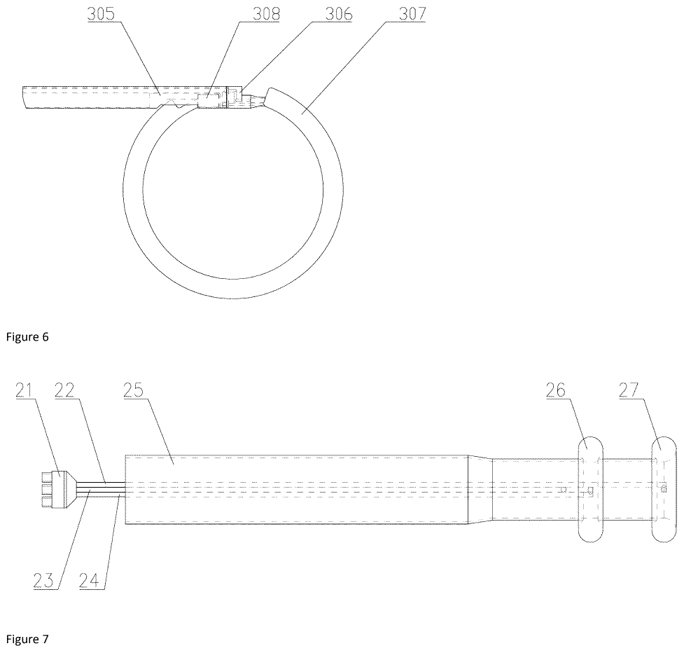

FIG. 3 shows a side view of the inflatable colorectal outer wall constriction device. As shown in FIG. 3, in some embodiments, the outer diameter of the support rod (302) is larger than the outer diameter of the coiled inflatable balloon (307), and the outer diameter of the coiled inflatable balloon (307) is larger than the outer diameter of the head inflatable balloon (308).

Referring to FIG. 7, in some embodiments, the fecal diversion device (2) includes an inflatable washing section (21), a first balloon tube (22), a cleaning tube (23), a second balloon tube (24), an intestinal protective membrane (25), a first support balloon (26), and a second support balloon (27). The intestinal protective membrane (25) has a hollow conduit, with one end of the first balloon tube (22), the cleaning tube (23), and the second balloon tube (24) located within the hollow conduit of the intestinal protective membrane (25), and the other end positioned outside the intestinal protective membrane (25) and the intestinal cavity, and connected to the opening of the inflatable washing section (21).

Referring again to FIG. 7, in some embodiments, the first support balloon (26) and the second support balloon (27) are spaced apart and sleeved on the end of the intestinal protective membrane (25) away from the inflatable washing section (21), with the first support balloon (26) connected to the first balloon tube (22) and the second support balloon (27) connected to the second balloon tube (24).

In some embodiments, the inflatable colorectal outer wall constriction device provided by this application is positioned between the first support balloon (26) and the second support balloon (27). The first support balloon (26) and the second support balloon (27) are constrained against the outer wall of the intestine by the inflatable colorectal outer wall constriction device to ensure tight sealing between the first support balloon (26) and the second support balloon (27) and the inner wall of the intestine.

As shown in FIG. 2, a portion of the fecal diversion device (2) is located outside the intestinal cavity (1), while another portion is located inside the intestinal cavity (1). Specifically, as shown in FIG. 2, the inflatable washing section (21) and the end of the intestinal protective membrane (25) near the inflatable washing section (21) are located outside the intestinal cavity (1), while the first support balloon (26), the second support balloon (27), and the inflatable colorectal outer wall constriction device (3) are located inside the intestinal cavity (1).

Referring again to FIG. 2, the intestinal protective membrane (25) has a hollow conduit, with the first balloon tube (22), the cleaning tube (23), and the second balloon tube (24) located within the hollow conduit of the intestinal protective membrane (25). The intestinal protective membrane (25) includes a first conduit and a second conduit, with the outer diameter of the first conduit being larger than that of the second conduit. The first conduit and the second conduit are connected through a connecting part. The first conduit is positioned closer to the inflatable washing section (21) compared to the second conduit, with part of the first conduit located outside the intestinal cavity (1) and the other part of the first conduit and the second conduit located inside the intestinal cavity (1). The first support balloon (26) and the second support balloon (27) have central through-hole structures, with the first support balloon (26) and the second support balloon (27) sleeved on the second conduit, and the second support balloon (27) located at the end of the second conduit away from the first conduit. The inflatable colorectal outer wall constriction device (3) is positioned between the first support balloon (26) and the second support balloon (27) to constrain the first support balloon (26) and the second support balloon (27) against the outer wall of the intestine.

In some embodiments, during use, the fecal diversion device (2) is inserted into the intestinal cavity through the anus, with part of the surface of the intestinal protective membrane (25) positioned outside the intestinal cavity.

In some embodiments, the opening of the inflatable washing section (21) includes a first balloon port, a second balloon port, and a cleaning port.

As shown in FIG. 2, in some embodiments, the first balloon port is connected to the first balloon tube (22), the second balloon port is connected to the second balloon tube (24), and the cleaning port is connected to the cleaning tube (23). The connections between the first balloon port and the first balloon tube (22), the second balloon port and the second balloon tube (24), and the cleaning port and the cleaning tube (23) are each provided with a one-way valve. The one-way valve can be opened during inflation and washing, and closed after use to ensure the first support balloon (26) and the second support balloon (27) remain sealed. The valves can be manually deflated for removal.

In some embodiments, the side wall of the intestinal protective membrane (25) is provided with cleaning holes that are connected to the cleaning tube (23).

In some embodiments, the cleaning holes are located at the position where the intestinal protective membrane (25) contacts the anastomosis site (11).

The method of using the device provided by this invention includes the following steps:
1. During use, the fecal diversion device (2) is inserted into the intestinal cavity (1) and inflated to secure it in place. The inflatable colorectal outer wall constriction device (3) is positioned between the two balloons at the front end of the fecal diversion device (2). The coiled balloon (307) of the inflatable colorectal outer wall constriction device (3) is inflated to form a coil, and the head balloon (308) is inserted into the slot (305) of the support rod (302) and then inflated to secure it, forming an elastic annular sealing structure.
2. For removal, the head balloon (308) and the coiled balloon (307) are deflated, the coiled balloon (307) straightens out with the help of the first elastic metal rod (309) and the second elastic metal rod (310), and the device is then removed from the body.

According to the above technical solutions, an embodiment of the present application provides an inflatable colorectal outer wall constriction device, which includes: a support rod (302), a coiled balloon tube (303), a head balloon tube (304), a coiled balloon (307), and a head balloon (308). One end of the support rod (302) is provided with an inflation port (301), and the other end is sequentially connected to the coiled balloon (307) and the head balloon (308). During use, the inflation port (301) is positioned outside the body. The inflation port (301) includes a first inflation port and a second inflation port. The support rod (302) has a hollow conduit, with the coiled balloon tube (303) and the head balloon tube (304) located within the hollow conduit. The first inflation port is connected to the coiled balloon (307) via the coiled balloon tube (303), and the second inflation port is connected to the head balloon (308) via the head balloon tube (304). The end of the support rod (302) near the coiled balloon (307) is provided with a slot (305) that matches the head balloon (308). When air is pumped into the first inflation port, the coiled balloon (307) can coil into an annular shape. When air is pumped into the second inflation port, the head balloon (308) expands and locks into the slot (305), thereby applying a certain constraint to the outer wall of the intestine to ensure a tight seal between the fecal diversion device (2) and the inner wall of the intestinal cavity (1).

The inflatable colorectal outer wall constriction device provided by this application achieves coiling and locking actions through a two-stage balloon mechanism. The constriction force can be adjusted by the inflation pressure. During dismantling, the device straightens out with the help of the internal elastic metal rods. Compared to existing solutions, this device increases the external pressure on the intestinal wall, providing better sealing and preventing residual feces from leaking and contaminating the anastomosis site. The coiling and straightening actions can be performed outside the body, making it safe and reliable. The device provided by this application has a simple structure, does not require special materials, and has lower costs, offering better economic efficiency compared to existing complex mechanical constriction mechanisms or absorbable materials.

It should be understood by those skilled in the art that the above embodiments are specific examples for implementing the present application. Various modifications and changes can be made in form and detail without departing from the spirit and scope of the present application. Therefore, the protection scope of the present application should be determined by the appended claims.

1. The inflatable colorectal outer wall constriction device, characterized in that it comprises: A support rod, a curling balloon airway, a head balloon airway, a curling balloon, and a head balloon. Wherein one end of the support rod is provided with an inflation port, and the other end is successively connected to the curling balloon and the head balloon. During use, the inflation port is located outside the body; the inflation port comprises a first inflation port and a second inflation port. The support rod has a hollow conduit, the curling balloon airway and the head balloon airway are located within the hollow conduit, and the first inflation port communicates with the curling balloon through the curling balloon airway, the second inflation port communicates with the head balloon through the head balloon airway. The end of the support rod near the curling balloon is provided with a slot matching the head balloon. When the first inflation port is inflated, the curling balloon can curl into a ring shape, and when the second inflation port is inflated, the head balloon expands, and the expanded head balloon is snapped into the slot, applying a certain constraint to the outer wall of the intestine to ensure a tight seal between the fecal diversion device and the inner wall of the intestine.

2. The inflatable colorectal outer wall constriction device according to claim 1, characterized in that the connection between the support rod and the curling balloon is provided with a balloon connecting part; the balloon connecting part is used to connect the support rod to the curling balloon.

3. The inflatable colorectal outer wall constriction device according to claim 1, characterized in that the side wall of the curling balloon is provided with a first elastic metal rod and a second elastic metal rod; When inflated, the curling balloon curls into a ring shape, and when deflated, the curling balloon naturally unfolds under the action of the first elastic metal rod and the second elastic metal rod.

4. The inflatable colorectal outer wall constriction device according to claim 3, characterized in that the inner wall of the curling balloon includes a first inner wall and a second inner wall, the thickness of the first inner wall being greater than the thickness of the second inner wall; The first elastic metal rod and the second elastic metal rod are embedded in the first inner wall.

5. The inflatable colorectal outer wall constriction device according to claim 4, characterized in that the head balloon airway penetrates the first inner wall and connects to the head balloon, and the head balloon airway passes between the first elastic metal rod and the second elastic metal rod; The first elastic metal rod and the second elastic metal rod are symmetrically arranged with respect to the head balloon airway.

6. The inflatable colorectal outer wall constriction device according to claim 3, characterized in that the cross-section of the first elastic metal rod and the second elastic metal rod is elliptical, and the first elastic metal rod and the second elastic metal rod can only bend and recover in one direction, ensuring that the curling balloon accurately curls into a ring shape when inflated and returns to a straight line when deflated.

7. The inflatable colorectal outer wall constriction device according to claim 1, characterized in that the outer diameter of the support rod is greater than the outer diameter of the curling balloon, and the outer diameter of the curling balloon is greater than the outer diameter of the head balloon.

8. The inflatable colorectal outer wall constriction device according to claim 1, characterized in that the fecal diversion device includes an inflation and irrigation part, a first balloon airway, a cleaning tube, a second balloon airway, an intestinal protective membrane, a first support balloon, and a second support balloon. Wherein the intestinal protective membrane has a hollow conduit, and one end of the first balloon airway, the cleaning tube, and the second balloon airway is located in the hollow conduit of the intestinal protective membrane, the other end is located outside the intestinal protective membrane and the intestine cavity, and the other end is connected to the opening of the inflation and irrigation part.

9. The inflatable colorectal outer wall constriction device according to claim 8, characterized in that the first support balloon and the second support balloon are spaced apart at the end of the intestinal protective membrane away from the inflation and irrigation part, and the first support balloon is connected to the first balloon airway, and the second support balloon is connected to the second balloon airway.

10. The inflatable colorectal outer wall constriction device according to claim 8, characterized in that the inflatable colorectal outer wall constriction device is located in the middle of the first support balloon and the second support balloon. The first support balloon and the second support balloon are constrained by the inflatable colorectal outer wall constriction device to ensure a tight seal between the first support balloon, the second support balloon, and the inner wall of the intestine.
